(19) United States
(12) Patent Application Publication
(10) Pub. No.: US 2020/0104733 A1
(43) Pub. Date: Apr. 2, 2020

(54) GENERATION OF HUMAN READABLE EXPLANATIONS OF DATA DRIVEN ANALYTICS

(71) Applicant: Palo Alto Research Center Incorporated, Palo Alto, CA (US)

(72) Inventor: Evgeniy Bart, Santa Clara, CA (US)

(21) Appl. No.: 16/143,802

(22) Filed: Sep. 27, 2018

(51) Int. Cl. G06N 5/04; G16H 50/20; G06N 3/02; G06N 99/00

(52) U.S. Cl. CPC G06N 5/045; G06N 99/005; G06N 3/02; G16H 50/20

(57) ABSTRACT

Systems and method describe inputting a set of characteristic data to a machine learning model that was trained at least in part on a knowledge based data set. A predicted outcome is determined based on the output of the machine learning model and a subset of the knowledge based data set that includes terms corresponding to the set of characteristic data is identified. The predicted outcome and subset of the knowledge based data set is used to generate display information for an interface.

### TECHNICAL FIELD

Implementations of the present disclosure relate to data analytics.

### BACKGROUND

The use of data analytics can improve the accuracy or diagnosis, identification, prognosis, or other predictions in a variety of environments. These techniques can include hard coded decision trees, automatic machine learning, deep learning, or other uses of data to provide a predictive outcome.

### BRIEF DESCRIPTION OF THE DRAWINGS

The described embodiments and the advantages thereof may best be understood by reference to the following description taken in conjunction with the accompanying drawings. These drawings in no way limit any changes in form and detail that may be made to the described embodiments by one skilled in the art without departing from the spirit and scope of the described embodiments.

FIG. 1 is a schematic diagram of an embodiment of systems to analyze data, which can be used in accordance with some embodiments.

FIG. 2 is an example interface generated by an analytics system, in accordance with some embodiments.

FIG. 3 is a flow diagram of an embodiment of a method of applying a machine learning model, in accordance with some embodiments.

FIG. 4 is a flow diagram of an embodiment of a method of applying a machine learning model, in accordance with some embodiments.

FIG. 5 is an illustration showing an example computing device which may implement the embodiments described herein.

### DETAILED DESCRIPTION

Described herein are systems that automatically generate human understandable explanations for the output of machine learning systems. Machine learning approaches to data analytics can provide insight and solutions that are not previously known in a field. For example, a machine learning system can be trained on a large set of data to identify connections between different elements in the set of data that are not obvious to a human. The insights developed through machine learning systems can replace or augment the available recorded data in particular fields. For example, a machine learning system can be used to predict medical diagnostics or outcomes based on previous patient data. The predicted diagnostics or outcomes can be provided to a medical practitioner to consider alongside expert knowledge and recorded medical texts.

However, while machine learning systems can provide additional or better information than an expert, the systems can also make mistakes. For example, a set of sample data may have few occurrences of certain characteristics. Because of the small sample size, those occurrences may have random co-occurrences that are given disproportionate weight. Small numbers of certain terms or characteristics in sample data used to train a machine learning model can create other mistakes as well. Furthermore, even when identifying accurate connections, there may be no clear reason why such connections exist. The lack of clarity as to why certain connections exist or certain outcomes are predicted by a machine learning system may not reduce the accuracy of the system, but it may reduce the adoption of the systems outputs by experts. For example, if a medical practitioner is given a predicted outcome that does not correlate with his expert opinion, then he may ignore the result of the machine learning system. However, assuming the machine learning system is accurate, this reduces the effectiveness of the system. Accordingly, described herein are systems and methods that generate human understandable explanations for outputs of machine learning systems.

For knowledge-driven systems (such as question-answering systems or classical expert systems) generating justification for an output can be performed due to the way these systems are built and trained. For example, the system can simply restate the question or rule that generated an output as the justification. However, such systems have several limitations. First, they are very labor-intensive and time-consuming to build. Second, they only encode existing formal knowledge (such as from medical textbooks or expert judgments) and do not encode new knowledge derived from data analysis. Because knowledge based data can have knowledge gaps, reliance on knowledge-driven systems can reduce the potential for analysis and output.

Machine learning systems can provide additional analysis that fills potential gaps in knowledge based data. A machine learning model can be trained on sample data for a field and then used by an analytics system to make predictions. However, these systems may offer no human-understandable indication of why a certain prediction is generated. When presented to an expert this can mean the expert will ignore the prediction if it does not follow an expected output based on the knowledge available to the expert.

Accordingly, analytics systems as described herein can use knowledge based data to provide an explanation for predictions generated by a machine learning model. The knowledge based data can be text data recording the knowledge available to an industry. For example, in the medical field, medical textbooks, journal articles, online resources, or other recordings of available knowledge can be used as knowledge based data. In some embodiments, new data is input into a machine learning model that is trained on sample data to generate a prediction. As discussed, the prediction may not include an indication of why it was generated from the new data. Based on the prediction, the analytics system can generate an alert or recommendation as well as provide the prediction. The analytics system can then search in the knowledge based data for explanatory statements that are consistent with the configuration of data that generated the prediction. The explanatory statements, potentially with a reference to the location of such statements in the knowledge based data, can be provided along with the predictions, alerts, or recommendations.

In some embodiments, the machine learning model can be a topic model trained on terms within the sample data. For example, each element in the sample data can include some description of the element. This characteristic data can include a number of terms that can be used to determine co-occurrences of terms within elements of the sample data. A machine learning training system can use the co-occurrences of these terms to generate groups of terms that have higher probabilities of co-occurrence with one another. After training, the machine learning model can be used to determine likely co-occurrences in new data and generate a prediction. Based on the prediction and the terms in the new data that contributed to that prediction, an analytics system can search in the knowledge based data for documents that include those terms. In some embodiments, the system can determine a particular document based on including those terms the most, the proximity of those terms within the document, the relationship of those terms within the document, or a combination of factors. One or more passages in the document can then be selected to explain the output of the machine learning model. For example, passages that include all of the relevant terms can be selected. The analytics system can then provide the passages as well as the prediction as an output.

In some embodiments, the knowledge based data can be used to augment training of the machine learning system. For example, a machine learning model can be trained using a set of sample data as well as a set of knowledge based data. In some embodiments, the knowledge based data can be analyzed to identify data or terms that correspond to those that appear in the sample data set. The identified terms or data can be organized into a format corresponding to the sample data. Thus, the knowledge based data can be used as a training input in the same manner as though it were part of the sample data. Using the knowledge based data as part of the training of the machine learning model can increase the likelihood of support for the output of the machine learning model. For example, if only sample data was used, there is a chance that there is no support in the knowledge based data from which to generate an explanatory description of the reasoning. However, if the knowledge based data is used during training, then the co-occurrence of terms or data in the knowledge based data is incorporated into the machine learning model.

In some embodiments, the machine learning model may be a topic model that can be used to identify likely co-occurrences in new set of characteristic data based on training to recognize co-occurrences in the sample data. In some embodiments the machine learning model can be a neural network that generates a prediction based on an input of characteristic data. For example, the neural network can be a recurrent neural network, a convolutional neural network, a feed forward neural network, a combination of layers of different types of neural network, or the like. In some embodiments, such neural networks may be trained using sample data or a combination of sample data and knowledge based data. In order to identify a human understandable explanation for the output of the neural network, the analytics system can search in the knowledge based data for the output prediction along with terms or data of the input characteristic data. The analytics system can then determine passages with the most correlation between the output prediction and the input characteristic data. For example, the search can require that the output prediction is present and search for documents with the most terms from characteristic data.

In other embodiments, additional types of machine learning models can be used to determine a prediction based on input characteristic data. The analytics system can then use the inputs, outputs, or both to determine a passage in the knowledge based system that explains the prediction. In some embodiments, the machine learning model may provide additional information about certain characteristic data that generated the prediction. For example, a topic model can provide an indication of which terms in the characteristic data were probabilistically associated with a prediction.

In some embodiments, when one or more passages or texts are identified as potentially explaining the reason for a prediction, the analytics system can select a preferred passage. For example, the passages may be preferred if they include the most reference to relevant terms, the best relationship between terms, the closest reference to certain terms, or may be selected based on a combination of factors. In some embodiments, multiple passages may be selected to explain the relationship between different characteristic data and the prediction. For example, two unrelated terms may each contribute to generating a prediction by the machine learning model. The analytics system can then identify separate passages describing that each of those terms contribute to the prediction and why.

In some embodiments, the analytics system may also track the results when different passages are provided. For example, after the prediction and explanation are provided to a user of the system, the system can track whether the prediction or recommendation is followed by the user. If it is not, the analytics system can track that a certain passage did not convince the expert to react to the prediction or recommendation. The analytics system can use such data when deciding between multiple passages to explain a prediction. For example, this can prevent the system from proving a passage that has routinely failed to explain certain predictions and instead provide another passage that may work better. In some embodiments, the track record for passages may be stored as weights for those passages when comparing at a later time.

In some embodiments, the machine learning model may create a prediction that is unsupported in the knowledge based data. In some embodiments, the analytics system can respond by providing the prediction without an explanatory statement of why the prediction occurred. In some embodiments, the analytics system may instead provide a degree of certainty or probability of the prediction that can convince a user to use the prediction or recommendation. In some embodiments, the analytics system may determine not to provide the prediction if support is not present in the knowledge based data. For example, this may prevent providing predictions that were generated based on spurious co-occurrences in the sample data.

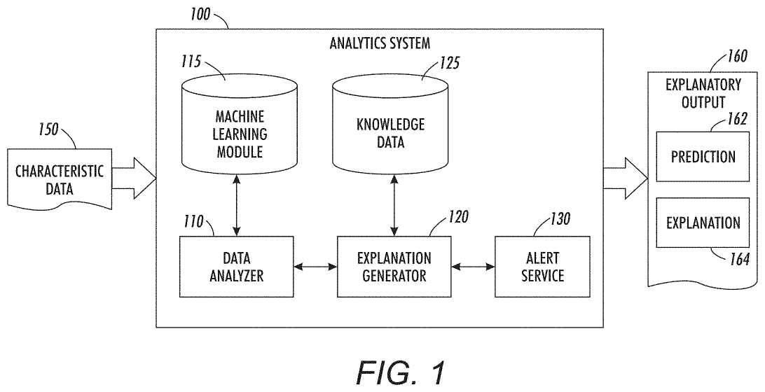

FIG. 1 is a diagram showing an analytics system 100. The analytics system 100 generates an explanatory output 160 based on characteristic data 150. For example, in a medical context, the characteristic data may be an admissions chart for a new patient at a hospital. The characteristic data can include diagnosis codes, procedure codes, personal information, or other data generated for new patients at a hospital. The analytics system 100 can then use the characteristic data to generate an explanatory output 160. The explanatory output 160 can include a prediction 162 generated by a data analyzer 110 as well as an explanation 164 for why or how the prediction was generated.

In some embodiments, the analytics system 100 includes a data analyzer 110, an explanation generator 120 and an alert service 130. The analytics system 100 can also include a machine learning model 115 and knowledge data 125. The machine learning model 115 can be used by the data analyzer 110 to generate a prediction based on characteristic data 160. The knowledge data 125 can be used by an explanation generator 120 to generate an explanation 164 for a prediction 162 that is generated by a data analyzer 110. In some embodiments, the knowledge data 125 may be stored at a remote location and accessed over a network or other connection by analytics system 100.

In order to generate a prediction based on characteristic data 150, the data analyzer 110 can input at a portion of the characteristic data 150 into the machine learning model 115. The machine learning model can then generate an output. In some embodiments, the output of the machine learning model can be a prediction of whether or not a particular outcome will occur. In some embodiments, the output can be a probability of the occurrence of one or more outcomes. For example, if the machine learning model is a topic model representing the probability of co-occurrence of certain items then the output may be an inference of the likelihood of certain other items in response to the input of characteristic data. The data analyzer 110 can use the probabilities provided by the machine learning model 115 to generate a prediction of which of the items are most likely to occur. For example, in the medical context, the characteristic data 150 can include a set of diagnosis and procedure codes. The data analyzer 110 can input those codes into the machine learning model 115 and receive the probability that other codes that were not in the characteristic data 150 will co-occur based on the codes. The data analyzer 110 can then select one or more of those codes to generate a prediction. In some embodiments, the data analyzer 110 can provide a prediction based on the most likely code, based on a priority level associated with certain codes (e.g., for serious conditions), based on the probability of a code being above a threshold value, or based on other criteria. The prediction generated by the data analyzer 110 can then be provided to an explanation generator 120.

In some embodiments, the machine learning model 115 may be a topic model that associates terms in characteristic data 150 with one another based on the probability of co-occurrences of those terms in a set of sample data. In some embodiments, the machine learning model 115 may have been trained based on sample data as well as knowledge data 125. For example, the combination of the sample data from measured or observed occurrences can be augmented by additional knowledge in the knowledge data 125. Training the model as a topic model can be performed with modifications of one or more of latent dirichlet allocation (LDA), probabilistic latent semantic analysis, or other topic modeling.

In some embodiments, the machine learning model 115 may be a neural network that is trained based on a set of sample data that is tagged with one or more outcomes. For example, the sample data can have one or more flags for different outcomes and the machine learning model can be trained to predict those outcomes based on the inputs of new data. In some embodiments, additional machine learning techniques can be used. For example, auto-encoders can be used to map characteristic data to a latent space where it can be analyzed in relation to its position in that space.

The predictive model 115 can be used by the data analyzer 110 to predict outcomes based on characteristic data 150. The analytics system 100 can receive characteristic data 150 from an internal or external system. For example, the characteristic data 150 could be hospital admission data for a new patient. In some embodiments, the analytics system 100 can be hosted on the same computer system as a system that receives the characteristic data 150, or it can be hosted on a different device. In some embodiments, the analytics system 100 can be hosted on a personal computer, laptop, tablet, phone, or other computing device in a room of a hospital where an output can be provided to a medical practitioner.

The data analyzer 110 can apply the machine learning model 115 to the received characteristic data 150 in order to generate a prediction 162. In some embodiments, the machine learning model is a neural network that receives characteristic data 150 and extracts features to generate an output. In some embodiments, the machine learning model 115 is a topic model that includes groups of terms in a number of topics. To apply the machine learning model 115, the data analyzer 110 can extract terms present in the characteristic data 150 and identify additional terms that may be related to the characteristic data 150. For example, for a term in the characteristic data 150, there can be a corresponding term associated with one or more topics of the machine learning model 115. Based on a probability with which that term is associated with the topic, the data analyzer 110 can determine other related terms. As applied to each of the terms in the characteristic data 150, the analytics system can generate a set of terms that are predicted to also be associated with the source of the characteristic data. This can be used to generate a prediction 162.

In the context of medical diagnostics, the characteristic data 150 can include a number of diagnosis, procedures, or other information about a patient. For example, in some embodiments, the information can be received through admission data at a hospital. The data analyzer 110 can then use the machine learning model 115 to determine other diagnosis, procedures, or outcomes that have high probability of co-occurrence with the characteristic data 150 for the patient. For example, if the characteristic data 150 indicates a high probability of co-occurrence with a readmission, death, heart attack, or other negative consequence, that outcome can be provided to a medical practitioner to provide guidance for further treatment of the patient. In some embodiments, the prediction 162 can provide predicted outcomes, potentially related conditions, or other information. In some embodiments, the data analyzer 110 can provide as a prediction an alert for high risk patients or as an indication of the likelihood of certain events. For example, the data analyzer 110 can provide as a prediction a probability that a patient will be readmitted to the hospital within a period of time based on the application of the machine learning model 115 to the characteristic data 150.

The explanation generator 120 can use the prediction 162 generated by the data analyzer 110 to generate an explanation of why the prediction 162 was generated. For example, the explanation generator 120 can generate a human understandable explanation of why a particular prediction 162 was generated by a data analyzer 110. In some embodiments, the explanation generator 120 accesses the knowledge data 125 to identify a subset of the knowledge data 125 that can explain the prediction 162. In some embodiments, the explanation generator 120 also accesses the internal data structures of the data analyzer 110 to better identify the relevant subset of the knowledge data 125. For example, the explanation generator 120 can identify one or more passages based on the prediction 162, a portion of characteristic data 150, the internal state of the data analyzer 110, or a combination of the three to identify a portion of the knowledge data 125 to use as an explanation.

The knowledge data 125 can include records of knowledge in a relevant industry. For example, in the medical context, knowledge data 125 can include medical textbooks, journal articles, internet articles, or other data sources that include a record of knowledge about a certain field. In some embodiments, the knowledge data 125 can include other records of knowledge available to a field.

In some embodiments, the explanation generator 120 can search for terms in the characteristic data 150, the prediction 162, and the internal state of data analyzer 110 to identify passages in the knowledge data 125. For example, the explanation generator 120 can perform a search to identify documents in the knowledge data 125 that include the terms in the characteristic data 150 that lead to the prediction 162 by the data analyzer 110. In some embodiments, the explanation generator 120 may identify a number of documents or passages that include terms used by the data analyzer 110. The explanation generator 120 can then select one or more of the documents to use as an explanation based on the number or frequency of occurrences of the terms within those documents. In some embodiments, the explanation generator 120 can also perform textual analysis to determine which of multiple documents to provides as an explanation. For example, structural rules using sentence templates can be used to identify those documents with a highly correlated set of terms. As one example, documents that have terms related with “cause,” “effect,” “leads to,” or the like between the terms may be given a higher weight than those that simply list or include the words.

In some embodiments, the explanation generator 120 identifies particular passages within one or more documents in the knowledge data 125 to use as an explanation. For example, after identifying a document within the knowledge data 125, the explanation generator 120 can select a passage including the relevant terms to provide as an explanation. In some embodiments, the explanation generator 120 may select between multiple passages based on the differences between relationships of terms within those passages. In some embodiments, the explanation generator 120 may select different passages, potentially from different documents, in the knowledge data 125 to provide as an explanation. For example, there could be multiple terms in characteristic data 150 that contribute to the prediction 160, but are not related to one another. Accordingly, the explanation generator 120 may select a first document or passage associated with a first term and a second document or passages associated with the second term. In some embodiments, each of these terms may be provided as an explanation.

In some embodiments, the explanation generator 120 may store the response of users to particular passages or documents. For example, after the explanation is provided to a user with a prediction, the following procedures or action may indicate whether or not the prediction, recommendation, or alert was followed by the user. Based on the response, the explanation generator 120 can determine whether the explanation was effective. The explanation generator 120 can use the stored records of these results to weight documents or passages as more or less persuasive. For example, if two passages have been identified as explanations for a prediction, the explanation generator may determine that one has been more effective than the other in the past and provide the more effective passage as an explanation.

In some embodiments, other techniques can be used to identify an explanation for a prediction generated by the data analyzer 120. For example, a combination of weighting different factors or passages within a document can be used to optimize the selection of different passages. Furthermore, in addition to providing an explanation, in some embodiments the explanation generator 120 may also provide links or references to the source of the explanation or additional passages. Thus, a user may select different passages if a first explanation provided by the system does not explain the prediction.

In some embodiments, the analytics system 100 can also include an alert service 130. The alert service 130 may provide the explanatory output 160 in the form of active “push” alerts, rather than passive “pull” output. In other words, the system proactively informs the relevant party if certain conditions are met, as opposed to waiting for the human operator to perform a query on the system to obtain the prediction. For example, in a medical context, the system might proactively inform the attendant physician if the patient is at a high risk for readmission rather than wait for the physician to inquire as to the patient's risk. In some embodiments, the alert service 130 may provide the prediction 162 and the explanation 164 generated by the data analyzer 110 and explanation generator 120 as an explanatory output 160. The alert service 130 may provide an explanatory output 160 in response to all characteristic data 150 input into the analytics system 100 or may provide an explanatory output 160 only in cases where the prediction has a probability over a threshold or a criticality over a threshold. For example, in a medical context, only predicted diagnosis with an urgent threat to a patient may be provided as a prediction 162.

In some embodiments, the analytics system 100 can provide the explanation output 160 in a computer application, an email, automated text or telephone calls, printed output on admission charts for the patient, or in other formats through which to inform a medical practitioner of the output. While the outputs are discussed with respect to medical environments, in other fields additional relevant outputs could be provided. For example, the likelihood of a mechanical failure in a system, likelihood of student success at a college, or other predictions to inform an expert of the analysis results.

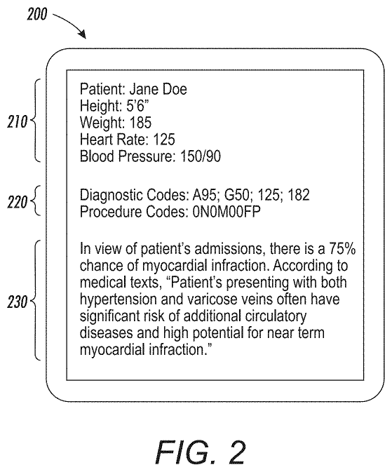

FIG. 2 illustrates an example interface 200 generated in response to a predictive outcome for a medical admission. In some embodiments, the example interface 200 can include admission information 210, diagnostic and procedure codes 220, and a predictive outcome 230 that includes a human understandable explanation for the prediction. As described with reference to FIG. 1, one or more of the admission information 210 and the diagnostic and procedure codes 220 can be used as characteristic data 150 by an analytics system 100. For example, in some embodiments, the analytics system 100 can apply the machine learning model 115 to the diagnostic and procedure codes 220 to generate a prediction about an outcome for a patient.

In some embodiments, only portions of the admission information 210 and the diagnostic and procedure codes 220 may be used to generate an outcome. Furthermore, in some embodiments, the data in the admission information 210 can be modified as an input to a machine learning model. For example, a patient's blood pressure can be characterized as high or low and used as a characteristic to provide to the machine learning model. Other characterizations can also be made such as calculating a person's body mass index, high or low heart rate, or other data for input to the machine learning model. Furthermore, in some embodiments, only the diagnostic or procedure codes 220 may be provided to the machine learning model.

Based on an output of a machine learning model based on the admission information 210 or the diagnostic and procedure codes 220, an analytics system can provide an indication 230 of a potential outcome for a patient. As shown in interface 200, the example prediction is that there is 75% chance of a myocardial infarction for the patient. As presented to a medical practitioner, this can help convince the medical practitioner to further analyze the medical records of the patient, keep the patient admitted, perform additional tests, or otherwise improve or change treatment of a patient. In some embodiments, fewer or additional details may be provided than as shown in the interface 200. For example, in some embodiments, the probability of an outcome may not be provided and instead just an indication to perform further tests can be given. Furthermore, in some embodiments, additional potential diagnosis or recommended procedures can be provided as part of the predicted outcome 230.

The predicted outcome 230 also includes an explanation for why the prediction was generated by the machine learning model. For example, as shown in interface 200, “Patient's presenting with both hypertension and varicose veins often have significant risk of additional circulatory diseases and high potential for near term myocardial infarction.” The explanation may have been generated from a medical text book based on analysis of the prediction and diagnosis codes. For example “hypertension” and “varicose veins” may be identified terms based on the diagnosis codes 220. Based on input of all of the diagnosis codes and procedure codes 220 or the admission information 210, the machine learning model may identify related terms with a probability of occurrence. In this case, the machine learning model may identify a 75% chance of myocardial infarction for the patient. The analytics system can then search in a set of knowledge based data for documents or passages including the relevant terms. For example, here, the passage includes reference to diagnostic codes for hypertension and varicose veins along with the prediction “myocardial infarction.” In some embodiments, the analytics system may also identify other documents or passages that also include the identified terms. The analytics system may then select the shown passage based on length, textual analysis, or other information in the passages or about success rates of the passages. In some embodiments, the analytics system may have determined that the diagnosis codes for hypertension and varicose veins were those that contributed to the prediction as opposed to other diagnosis codes in the characteristic data.

In various embodiments, the interface 200 may include fewer or additional elements than shown in FIG. 2. For example, in some embodiments, the prediction and explanation may be provided without diagnosis codes 220 or admission information 210. The interface 200 could also include links or reference information for the source of the explanation. Furthermore, in some embodiments, the interface 200 may be provided in different formats or on different mediums. For example, the interface 200 may be provided over text, email, user interface, automated audio, or other formats that can provide the prediction and explanation.

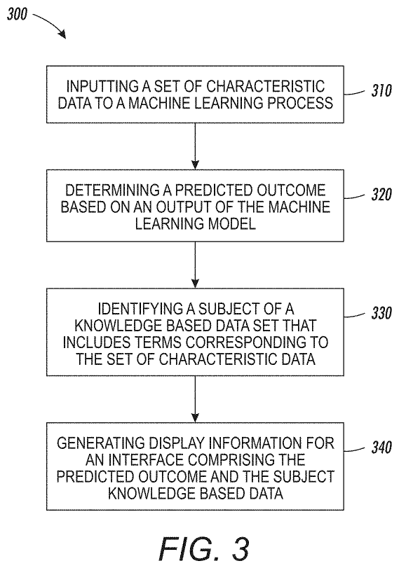

FIG. 3 is a flow chart 300 illustrating example operations of an analytics system generating a prediction with a machine learning model and a human understandable explanation for the prediction. For example, the processes described with reference to FIG. 3 may be performed by an analytics system 100 as described with reference to FIG. 1. Beginning at block 310, an analytics system inputs a set of characteristic data to a machine learning model. In some embodiments, the machine learning model was trained using both a sample data set and a knowledge based data set. For example, the machine learning model may be trained on medical admissions data and a set of medical texts.

In block 320, the analytics system may determine predicted outcome based on an output of the machine learning model. In some embodiments, the machine learning model may provide the predicted outcome. For example, the machine learning model may be a neural network that provides a prediction based on data within the characteristic data. In some embodiments, the output of the machine learning model may include probabilities of co-occurrence of additional terms. The analytics system may predict an outcome based on an inference of which of these terms are most likely. In some embodiments the analytics system may prioritize predictions based on criticality of terms in addition to likelihood. For example, if one potential outcome is more likely, but less important, then the analytics system may provide a less likely but more severe potential outcome as the predicted outcome.

In block 330, the analytics system can identify a subset of a knowledge based data set that includes terms corresponding to the set of characteristic data. In some embodiments, the knowledge based data set may be the same or similar to one that was used to at least in part train the machine learning system. In some embodiments, the knowledge based data set may be separate from any data set that was used to training the machine learning model. In some embodiments, the subset of the knowledge set may include one or more passages from one or more documents of the knowledge based data set. In some embodiments, the identified subset may also be selected as including terms related to the predicted outcome in addition to terms that contributed to the prediction from the characteristic data.

In block 340, the analytics system generates display information for an interface comprising the predicted outcome and the subset of the knowledge based data set. For example, in some embodiments, display information to be displayed on an interface such as interface 200 in FIG. 2 may be generated by the analytics system. In some embodiments, the interface may include fewer or additional features. For example, the interface may include a selected passage as shown in interface 200 but also reference or links to the documents from which the passage was selected. In some embodiments, the analytics system may generate other interfaces including additional information or functionality.

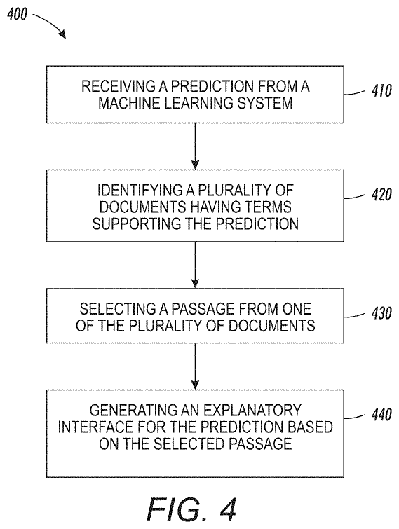

FIG. 4 is a flow chart 400 illustrating example operations of an analytics system. For example, the processes described with reference to FIG. 4 may be performed by an analytics system 100, or portion thereof, as described with reference to FIG. 1. Beginning at block 410, the analytics system receives a predicted outcome generated by a machine learning system based on a set of characteristic data. In some embodiments, the predicted outcome may also be based on the machine learning system's internal state. The characteristic data may be in a same or similar format to one that was used to train the machine learning system. In some embodiments, the machine learning system was also trained using a set of knowledge based data.

In block 420, the analytics system identifies, from a set of knowledge based data, a plurality of documents having terms from the set of characteristic data, the predicted outcome, and the machine learning system's internal state. For example, the identified documents may each have at least a threshold number of terms from the characteristic data and the predicted outcome. In some embodiments, the identified documents may be selected to include the predicted outcome as well as one of the terms that contributed to the selection of the predicted outcome. In some embodiments, the analytics system may identify documents that have the largest number of terms from the characteristic data. For example, some machine learning models may not indicate which terms in the characteristic data contributed to selecting the predicted outcome. Accordingly, documents may be selected that use the most terms, the highest frequency of terms, or the most instances of terms. In some embodiments, a single document may be selected from the set of documents.

In block 430, the analytics system selects a passage from one of the plurality of documents. In some embodiments, the analytics system can select multiple passages and those may be from multiple documents of the plurality of documents. In some embodiments, the passages may be selected based on textual analysis of the relationship between terms. The passages may also be selected based on the proximity of terms, the number of terms within a section of text, or other criteria. In some embodiments, the analytics system may also keep track of the results after a passage is selected and use such results to weight those passages for future selection.

In block 440, the analytics system generates an interface comprising the predicted outcome and the subset of the knowledge based data set. For example, in some embodiments, an interface such as interface 200 in FIG. 2 may be generated by the analytics system. In some embodiments, the interface may include fewer or additional features. For example, the interface may include a selected passage as shown in interface 200 but also reference or links to the documents from which the passage was selected. In some embodiments, the analytics system may generate other interfaces including additional information or functionality.

Various operations are described as multiple discrete operations, in turn, in a manner that is most helpful in understanding the present disclosure, however, the order of description may not be construed to imply that these operations are necessarily order dependent. In particular, these operations need not be performed in the order of presentation.

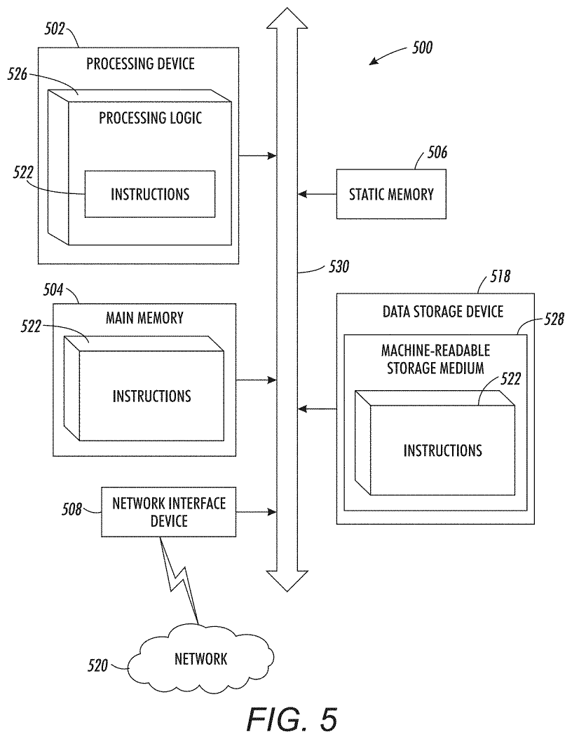

FIG. 5 illustrates a diagrammatic representation of a machine in the example form of a computer system 500 within which a set of instructions, for causing the machine to perform any one or more of the methodologies discussed herein, may be executed. In alternative embodiments, the machine may be connected (e.g., networked) to other machines in a local area network (LAN), an intranet, an extranet, or the Internet. The machine may operate in the capacity of a server or a client machine in a client-server network environment, or as a peer machine in a peer-to-peer (or distributed) network environment. The machine may be a personal computer (PC), a tablet PC, a set-top box (STB), a Personal Digital Assistant (PDA), a cellular telephone, a web appliance, a server, a network router, a switch or bridge, a hub, an access point, a network access control device, or any machine capable of executing a set of instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein. In one embodiment, computer system 500 may be representative of a server computer system, such as analytics system 100 or analytics system 160.

The exemplary computer system 500 includes a processing device 502, a main memory 504 (e.g., read-only memory (ROM), flash memory, dynamic random access memory (DRAM), a static memory 506 (e.g., flash memory, static random access memory (SRAM), etc.), and a data storage device 518, which communicate with each other via a bus 530. Any of the signals provided over various buses described herein may be time multiplexed with other signals and provided over one or more common buses. Additionally, the interconnection between circuit components or blocks may be shown as buses or as single signal lines. Each of the buses may alternatively be one or more single signal lines and each of the single signal lines may alternatively be buses.

Processing device 502 represents one or more general-purpose processing devices such as a microprocessor, central processing unit, or the like. More particularly, the processing device may be complex instruction set computing (CISC) microprocessor, reduced instruction set computer (RISC) microprocessor, very long instruction word (VLIW) microprocessor, or processor implementing other instruction sets, or processors implementing a combination of instruction sets. Processing device 502 may also be one or more special-purpose processing devices such as an application specific integrated circuit (ASIC), a field programmable gate array (FPGA), a digital signal processor (DSP), network processor, or the like. The processing device 502 is configured to execute processing logic 526, which may be one example of system 400 shown in FIG. 4, for performing the operations and steps discussed herein.

The data storage device 518 may include a machine-readable storage medium 528, on which is stored one or more set of instructions 522 (e.g., software) embodying any one or more of the methodologies of functions described herein, including instructions to cause the processing device 502 to execute analytics system 100 or analytics system 160. The instructions 522 may also reside, completely or at least partially, within the main memory 504 or within the processing device 502 during execution thereof by the computer system 500; the main memory 504 and the processing device 502 also constituting machine-readable storage media. The instructions 522 may further be transmitted or received over a network 520 via the network interface device 508.

The machine-readable storage medium 528 may also be used to store instructions to perform a method for analyzing log data received from networked devices, as described herein. While the machine-readable storage medium 528 is shown in an exemplary embodiment to be a single medium, the term “machine-readable storage medium” should be taken to include a single medium or multiple media (e.g., a centralized or distributed database, or associated caches and servers) that store the one or more sets of instructions. A machine-readable medium includes any mechanism for storing information in a form (e.g., software, processing application) readable by a machine (e.g., a computer). The machine-readable medium may include, but is not limited to, magnetic storage medium (e.g., floppy diskette); optical storage medium (e.g., CD-ROM); magneto-optical storage medium; read-only memory (ROM); random-access memory (RAM); erasable programmable memory (e.g., EPROM and EEPROM); flash memory; or another type of medium suitable for storing electronic instructions. In some embodiments, the data storage device, the memory, the network, the processing device, and other components may store and/or access the data, including the training data for the machine learning model and the knowledge data for the explanation generator. This data may be stored in raw form or in a preprocessed form, depending on the application.

The preceding description sets forth numerous specific details such as examples of specific systems, components, methods, and so forth, in order to provide a good understanding of several embodiments of the present disclosure. It will be apparent to one skilled in the art, however, that at least some embodiments of the present disclosure may be practiced without these specific details. In other instances, well-known components or methods are not described in detail or are presented in simple block diagram format in order to avoid unnecessarily obscuring the present disclosure. Thus, the specific details set forth are merely exemplary. Particular embodiments may vary from these exemplary details and still be contemplated to be within the scope of the present disclosure.

Additionally, some embodiments may be practiced in distributed computing environments where the machine-readable medium is stored on and or executed by more than one computer system. In addition, the information transferred between computer systems may either be pulled or pushed across the communication medium connecting the computer systems.

Embodiments of the claimed subject matter include, but are not limited to, various operations described herein. These operations may be performed by hardware components, software, firmware, or a combination thereof.

Although the operations of the methods herein are shown and described in a particular order, the order of the operations of each method may be altered so that certain operations may be performed in an inverse order or so that certain operation may be performed, at least in part, concurrently with other operations. In another embodiment, instructions or sub-operations of distinct operations may be in an intermittent or alternating manner.

The above description of illustrated implementations of the invention, including what is described in the Abstract, is not intended to be exhaustive or to limit the invention to the precise forms disclosed. While specific implementations of, and examples for, the invention are described herein for illustrative purposes, various equivalent modifications are possible within the scope of the invention, as those skilled in the relevant art will recognize. The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form. Moreover, use of the term “an embodiment” or “one embodiment” or “an implementation” or “one implementation” throughout is not intended to mean the same embodiment or implementation unless described as such. Furthermore, the terms “first,” “second,” “third,” “fourth,” etc. as used herein are meant as labels to distinguish among different elements and may not necessarily have an ordinal meaning according to their numerical designation.

It will be appreciated that variants of the above-disclosed and other features and functions, or alternatives thereof, may be combined into may other different systems or applications. Various presently unforeseen or unanticipated alternatives, modifications, variations, or improvements therein may be subsequently made by those skilled in the art which are also intended to be encompassed by the following claims. The claims may encompass embodiments in hardware, software, or a combination thereof.

What is claimed is:

1. A method comprising:
inputting a set of characteristic data to a machine learning model;
determining a predicted outcome based on an output of the machine learning model;
identifying a subset of a knowledge based data set that explains the predicted outcome; and
generating display information for an interface, the display information comprising the predicted outcome and the subset of the knowledge based data set.

2. The method of claim 1, wherein identifying the subset of the knowledge based data comprises:
determining a first term in the characteristic data that is associated with the predicted outcome; and
identifying a passage in the knowledge based data set that includes the first term and the predicted outcome.

3. The method of claim 1, wherein identifying the subset of the knowledge based data comprises:
determining a first term and a second term contributing to the determination of the predicted outcome; and
identifying a passage in the knowledge based data set that includes the first term and the second term.

4. The method of claim 1, wherein identifying the subset of the knowledge based data set comprises determining a term in the set of characteristic data that is grouped with the predicted outcome in a topic model.

5. The method of claim 1, wherein the machine learning model is one of a topic model or a neural network.

6. The method of claim 1, wherein identifying the subset of the knowledge based data set comprises:
identifying a first passage of the knowledge based data set that includes the terms corresponding to the set of characteristic data;
identifying a second passage of the knowledge based data set that includes the terms corresponding to the set of characteristic data; and
selecting the first passage over the second passage based on a relationship of the terms within the first passage.

7. The method of claim 1, wherein the characteristic data is generated from medical admissions and the knowledge based data comprises medical texts.

8. The method of claim 1, wherein identifying a subset of the knowledge based data comprises:
identifying a first passage describing a relationship between a first term of the set of characteristic data and the predicted outcome; and
identifying a second passage describing a relationship between a second term of the set of characteristic data and the predicted outcome.

9. The method of claim 1, further comprising:
inputting a second set of characteristic data to the machine learning model;
determining a predicted outcome based on a second output of the machine learning model; and
in response to determining that no subset of the knowledge based data includes terms corresponding to the set of characteristic data and the predicted outcome, rejecting the predicted outcome.

10. A non-transitory computer-readable medium having instructions stored thereon that, when executed by a processing device, cause the processing device to:
input a medical admission record of a patient to a machine learning model to generate an output;
determine a predicted outcome based on the output of the machine learning model;
identify a subset of a set of medical texts that includes terms corresponding to at least some of the terms of the medical admission record; and
generating an interface comprising the predicted outcome and the subset of the set of medical texts.

11. The non-transitory computer-readable medium of claim 10, wherein to identify the subset of the set of medical texts, the processing device is further to:
determine a first term in the medical admission record that is associated with the predicted outcome; and
identify a passage in the set of medical texts that includes the first term and the predicted outcome.

12. The non-transitory computer-readable medium of claim 10, wherein to identify the subset of the set of medical texts, the processing device is further to:
determine a first term and a second term contributing to the determination of the predicted outcome; and
identifying a passage in the set of medical texts that includes the first term and the second term.

13. The non-transitory computer-readable medium of claim 10, wherein to identify the subset of the set of medical texts, the processing device is further to determine a term in the medical admission record that is grouped with the predicted outcome in a topic model.

14. The non-transitory computer-readable medium of claim 10, wherein the machine learning model is one of a topic model or a neural network.

15. The non-transitory computer-readable medium of claim 10, wherein to identify the subset of the set of medical texts, the processing device is further to:
identify a first passage of the set of medical texts that includes the terms corresponding to the medical admission record;
identify a second passage of the set of medical texts that includes the terms corresponding to the medical admission record; and
selecting the first passage over the second passage based on a relationship of the terms within the first passage.

16. The non-transitory computer-readable medium of claim 10, wherein to identify the subset of the set of medical texts, the processing device is further to:
identify a first passage describing a relationship between a first term of the medical admission record and the predicted outcome; and
identifying a second passage describing a relationship between a second term of medical admission record and the predicted outcome.

17. A system comprising:
a memory device; and
a processing device operatively coupled to the memory device, wherein the processing device is to:
receive a predicted outcome generated by a machine learning system based on a set of characteristic data;
identify, from a set of knowledge based data, a plurality of documents explaining the predicted outcome;
select a passage from one of the plurality of documents; and
generate an explanatory interface for the prediction based on the selected passage.

18. The system of claim 17, wherein to select a passage from one of the plurality of documents the processing device is further to:
determine a first term in the characteristic data that is associated with the predicted outcome; and
identify a passage in the plurality of documents that includes the first term and the predicted outcome.

19. The system of claim 17, wherein to identify the subset of the knowledge based data the processing device is further to:
determine a first term and a second term contributing to the determination of the predicted outcome; and
identify a passage in the knowledge based data set that includes the first term and the second term.

20. The system of claim 17, wherein the machine learning model is one of a topic model or a neural network.
